(19) United States
(12) Patent Application Publication
(10) Pub. No.: US 2026/0295279 A1
(43) Pub. Date: Oct. 1, 2026

(54) EMERGENCY THERAPY OFF FEATURE FOR MEDICAL DEVICES WITH MINIMAL USER INTERFACE

(71) Applicant: Medtronic, Inc., Minneapolis, MN (US)

(72) Inventors: Andrew T. Fried, St. Paul, MN (US); Jacob P. Komarek, Fridley, MN (US); Eric P. Minnerath, Minneapolis, MN (US)

(21) Appl. No.: 19/630,555

(22) Filed: Mar. 27, 2026

Related U.S. Application Data
(60) Provisional application No. 63/779,646, filed on Mar. 28, 2025.

(51) Int. Cl. A61N 1/372

(52) U.S. Cl. CPC A61N 1/37247

(57) ABSTRACT

Techniques of the present disclosure relate to emergency therapy off functionality included with implantable medical devices (IMDs) and associated external instruments such as rechargers, communicators, and patient remotes. An external instrument for use with an IMD of the present disclosure can include a housing with an interface element, at least one processor inside the housing, and instructions that, when executed on the at least one processor, cause the at least one processor to: detect a unique input applied to the interface element, wherein the unique input is associated with activating an emergency therapy off feature to disable therapy for a patient; verify that the external instrument is in at least one predefined operational state before activating the emergency therapy off feature; and instruct the IMD to disable therapy in response to verifying that the external instrument is in the at least one predefined operational state.

### CROSS-REFERENCE TO RELATED APPLICATION

This application claims the benefit of and priority to U.S. Provisional Patent Application No. 63/779,646 filed Mar. 28, 2025, the entire disclosure of which is incorporated by reference herein.

### FIELD

The present disclosure generally relates to medical devices. More particularly, the present disclosure relates to emergency therapy off functionality included with implantable medical devices and associated external instruments such as rechargers, communicators, and patient remotes.

### BACKGROUND

Implantable medical devices (“IMDs”), such as neurostimulators, are increasingly used to manage a variety of conditions by delivering electrical stimulation or targeted therapies to specific areas of the body. These devices are typically paired with external instruments, such as wireless rechargers and communicators, to enable essential functions like recharging and therapy modulation. A critical requirement for these systems is the ability for patients to quickly and reliably turn off therapy during emergencies such as uncomfortable stimulation or unexpected side effects. Regulatory bodies, including the Food and Drug Administration in the United States, mandate that patients must be provided with the necessary tools to safely disable therapy.

Currently, many IMD systems rely on external patient handsets or magnets to enable emergency therapy off functionality. However, these solutions present challenges. For example, requiring a magnet to be held continuously over the implant to disable therapy is cumbersome and may lead to a poor user experience. Similarly, reliance on patient handsets or communicators, which patients may not always carry with them, creates a risk of being unable to access the necessary emergency therapy off functionality when needed.

Some IMDs, such as those used for neuromodulation, include external instruments like wireless rechargers, communicators, and patient remotes for various functional purposes (e.g., charging, communicating changes in therapy to the IMD). These external instruments generally have minimal or low user interfaces, and specifically minimal input or output mechanisms for user interaction. For example, certain external instruments used with IMDs feature a small number of buttons or indicators that either input commands to the IMD (e.g., start recharging) or output a specific quality or characteristic of the IMD (e.g., IMD is in the recharging state). This poses design constraints for including additional features such as emergency therapy off functionality with these external instruments.

### SUMMARY

Non-limiting examples of the present disclosure provide an external instrument for use with an IMD. The external instrument can include a housing with an interface element, at least one processor inside the housing, and instructions that, when executed on the at least one processor, can cause the at least one processor to: detect a unique input applied to the interface element, wherein the unique input is associated with activating an ETO feature to disable IMD therapy for a patient; verify that the external instrument is in at least one predefined operational state before activating the ETO feature; and instruct the IMD to disable therapy in response to verifying that the external instrument is in the at least one predefined operational state.

Non-limiting examples of the present disclosure provide a medical treatment system. The system can include an IMD configured to provide a medical therapy for a patient, and an external instrument coupled to the IMD. The external instrument can include a housing with an interface element, at least one processor, and instructions that, when executed on the at least one processor, can cause the at least one processor to: detect a unique input applied to the interface element, wherein the unique input is associated with activating an ETO feature to disable IMD therapy for a patient; verify that the external instrument is in at least one predefined operational state before activating the ETO feature; and instruct the IMD to disable therapy in response to verifying that the external instrument is in the at least one predefined operational state.

Non-limiting examples of the present disclosure provide a method or a non-transitory computer-readable medium encoding instructions operable to cause at least one processor of an external instrument for use with an IMD to operate. The method or the instructions can include detecting a unique input applied to the interface element, with the unique input generally being associated with activating an ETO feature to disable IMD therapy for a patient; verifying that the external instrument is in at least one predefined operational state before activating the ETO feature; and instructing the IMD to: disable therapy in response to verifying that the external instrument is in the at least one predefined operational state, and transition to an IMD reset state after therapy is disabled with the ETO feature; or prevent activation of the ETO feature if the unique input is not detected within a predetermined time period.

The above summary is not intended to describe each illustrated example, every implementation, or each advantage of the subject matter hereof. The figures and the detailed description that follow more particularly exemplify various examples and the advantages they provide over conventional ETO solutions.

### BRIEF DESCRIPTION OF THE DRAWING

Subject matter hereof may be more completely understood in consideration of the following detailed description of various examples in connection with the accompanying figures, in which:

FIG. 1 is a block diagram for a system having an implantable medical device, an external instrument having an emergency therapy off system, and optionally a user device, according to examples of the present disclosure;

FIG. 2 is a block diagram for a recharger having an emergency therapy off system for use with IMDs, according to examples of the present disclosure;

FIG. 3 is a perspective view of a recharger having an emergency therapy off system for use with IMDs, according to examples of the present disclosure;

FIG. 4 is a first state diagram for a recharger with an emergency therapy off system, illustrating initiating an emergency therapy off feature from a closed loop charging state, according to examples of the present disclosure;

FIG. 5 is a second state diagram for a recharger with an emergency therapy off system, illustrating initiating an emergency therapy off feature from a charging locating state, according to examples of the present disclosure;

FIG. 6 is a third state diagram for a patient handheld or therapy remote with an emergency therapy off system, illustrating initiating an emergency therapy off feature from a programming state, according to examples of the present disclosure; and

FIG. 7 is a method of operating an emergency therapy off system for use with implantable medical devices, according to examples of the present disclosure.

While various examples are amenable to various modifications and alternative forms, specifics thereof have been shown by way of example in the drawings and will be described in detail. It should be understood, however, that the intention is not to limit the claimed inventions to the particular examples described. On the contrary, the intention is to cover all modifications, equivalents, and alternatives falling within the spirit and scope of the subject matter as defined by the claims.

### DETAILED DESCRIPTION

In light of issues described in the Background, the present disclosure describes systems and devices that integrate emergency therapy off functionality into external instruments having minimal or low user interface (“UI”) such as certain wireless rechargers, communicators, and patient remotes. The present disclosure also describes methods of operating these systems or devices to enable emergency therapy off functionality with these external instruments. The present disclosure describes and illustrates these examples along with various advantages they provide over conventional solutions.

Examples of the present disclosure integrate an emergency therapy off (“ETO”) system into external instruments having minimal or low UI. These external instruments are typically used with a variety of IMDs including implantable neurostimulators. By integrating the ETO functionality into existing low-UI instruments, like wireless rechargers and communicators, examples of the present disclosure can eliminate or significantly reduce the need for a dedicated patient therapy remote system at all times. In some examples, the ETO system can be activated through a unique sequence of inputs, such as button presses, when the external instrument is in specific operational states to ensure it is not accidentally triggered during normal use. This simplifies the patient experience while maintaining safety, usability, and compliance with requirements to include an ETO system with certain IMDs. Accordingly, the present disclosure enables patients to safely and intuitively access ETO functionality using external instruments having low-UI (e.g., minimal input mechanisms such as buttons or output mechanisms such as indicators).

Advantages of the present disclosure include enabling patients to quickly turn off therapy during emergency events using external instruments that conventionally have not had this ability; eliminating the need for a magnet continuously held against the IMD to disable therapy; reducing the number of devices patients need to control their IMD(s); allowing patients to disable (and potentially enable) therapy when away from a primary IMD control device such as a patient handset or a communicator; designing the ETO system to be hidden behind specific input or button sequences during certain operational states to reduce the risk of accidental activation during normal use; designing the ETO system to work with a single input mechanism rather than multiple mechanisms or complex interfaces; making the ETO system compatible with existing external instruments including wireless rechargers, communicators, and patient remotes; providing broad compatibility across a range of IMDs and therapies including electrical stimulation and targeted drug delivery systems; and representing a simpler, more streamlined approach to implementing an ETO system that is also more cost effective for patients compared to conventional solutions.

Examples of the present disclosure include emergency therapy off (“ETO”) systems and devices, and methods for operating such systems and devices to implement ETO functionality with minimal or low UI external instruments, such as wireless rechargers, communicators, and patient remotes commonly used with implantable medical devices (“IMDs”). For IMDs such as neurostimulators, it is important for a patient to have the ability to stop therapy quickly in the event of an emergency or another situation that requires therapy to be turned off. This requirement exists because certain neurostimulators are implanted in the body and operate by stimulating the patient’s nervous system which in rare circumstances may cause pain or other side effects.

Certain IMD applications provide a patient with a handset device (e.g., smartphone, tablet) with custom patient software that connects to a communicator which acts as an intermediary between the software and the IMD. The communicator facilitates remote monitoring of the IMD, including transferring data to a healthcare provider to manage patient care. However, a patient occasionally may forget the handset device or the communicator when they leave home, or may not want to carry one or both devices with them when they travel. Currently, if the patient does not bring either device with them, they cannot turn off IMD therapy in the event of an emergency or another similar situation, and are unable to resume therapy if the IMD is turned off.

To remedy this, the present disclosure incorporates ETO functionality into low-UI external instruments like communicators, wireless rechargers, and patient remotes. The ETO functionality is integrated in these instruments by programming a unique sequence of inputs (e.g., pattern of button presses, button press(es) during certain operational states of the instruments) that, when performed by the patient, will execute the ETO feature to turn off IMD therapy. Using a unique sequence of inputs, optionally during specific operational states, will minimize accidental activation of the ETO system by the patient or others having access to the external instrument. Specific operational states contemplated by the present disclosure include the closed loop recharging or IMD locating states for wireless rechargers, and the idling or already IMD connected states for communicators. Accordingly, with a single button or interface element, the ETO system can be implemented with external instruments routinely used during IMD operation to stop therapy if an emergency event arises. Optionally, a similar feature can be included with the external instruments to turn therapy back on after the ETO system is used.

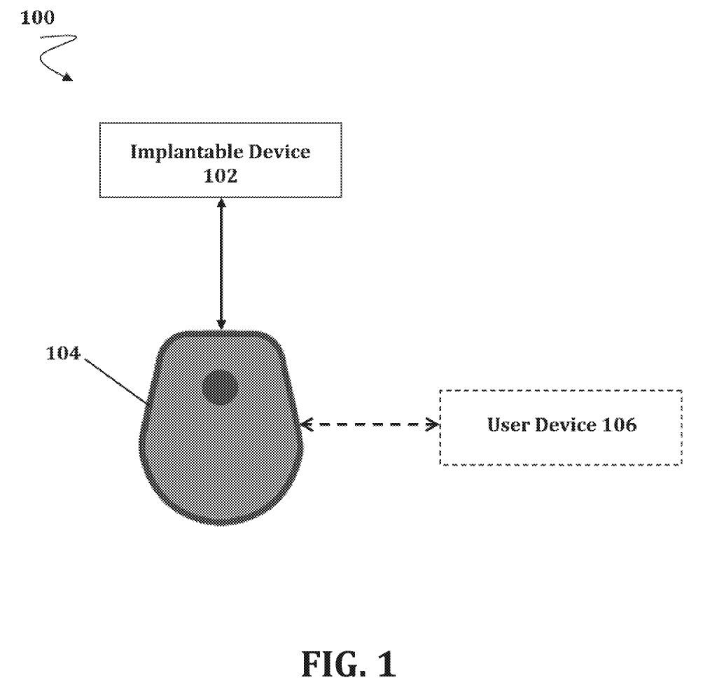

FIG. 1 is a block diagram for a system 100 having an emergency therapy off feature, according to examples of the present disclosure. System 100 generally includes an IMD 102, an external instrument 104 such as a recharger, and optionally, a user device 106. Although described herein as comprising a recharger, it should be understood that external instrument 104 can also comprise a communicator, a patient handheld or therapy remote, or another external instrument commonly used with IMD 102. For simplicity, external instrument 104 will be referred to as a recharger 104 in the following description. However, the communicator, remote, or other external instrument 104 may include the emergency therapy off functionality described herein for recharger 104 and other similar rechargers. In some examples, recharger 104, the communicator, and the patient remote can be combined as a single external instrument 104 configured to operate with IMD 102. In some examples, the external instrument 104 can be an electronic device such as a smartphone, tablet, wearable device like a smartwatch or a smart ring, among other similar devices. Certain external instruments 104 including electronic devices may comprise a digital patient application to control the IMD and activate the ETO system as needed.

In some examples, patient therapy using IMD 102 can have a short duty cycle. For example, the duty cycle where therapy is provided to the patient can be about 30 minutes during a 24-hour period, or about 30 minutes for three days a week. Other duty cycles less than or greater than these values are also contemplated by the present disclosure.

IMD 102 can include any medical device that can be surgically implanted or connected externally to a patient to treat medical conditions. In some examples, IMD 102 can include implantable neurostimulators (“INS”), drug delivery pumps, pacemakers, defibrillators, diagnostic recorders, or cochlear implants. In some examples, IMD 102 can include a battery that requires periodic charging or replenishing.

Recharger 104, which is one example of an external instrument 104, can be configured to replenish power in IMDs, such as IMD 102. In some examples, recharger 104 itself can include a battery that can be operably coupled to IMD 102 to replenish the battery of IMD 102. Accordingly, recharger 104 can itself be charged to replenish the battery of recharger 104, such as to an external power supply or a dock where recharger 104 can be received for charging. Additionally, recharger 104 can be configured to implement an ETO system to disable IMD therapy during emergencies or other situations that require therapy to stop. The patient or other user of recharger 104 can activate the ETO system by providing a unique input or input sequence, for example a unique button press arrangement or sequence, when recharger 104 is in specific operating states. This prevents accidental activation of the ETO system during normal operating circumstances.

In some examples, patients can be the primary users of recharger 104 and can coordinate recharging of IMD 102 with recharger 104. In some examples, clinicians can interface with recharger 104 to coordinate or alter recharging of IMD 102. In some examples, clinicians can update certain settings (e.g., volume of recharger 104 and recharger 104 speed or mode) through the interface.

Optional user device 106 can include a mobile platform such as a phone or tablet configured to run specialized software. For example, recharge application software can be hosted on user device 106. Recharge application software can be an application configured to communicate with recharger 104. Recharge application software can supplement the user experience of recharger 104. Generally, user device 106 can include a feature or command to disable IMD therapy in the case of emergencies, similar to the ETO system generally included with recharger 104. Examples of the present disclosure

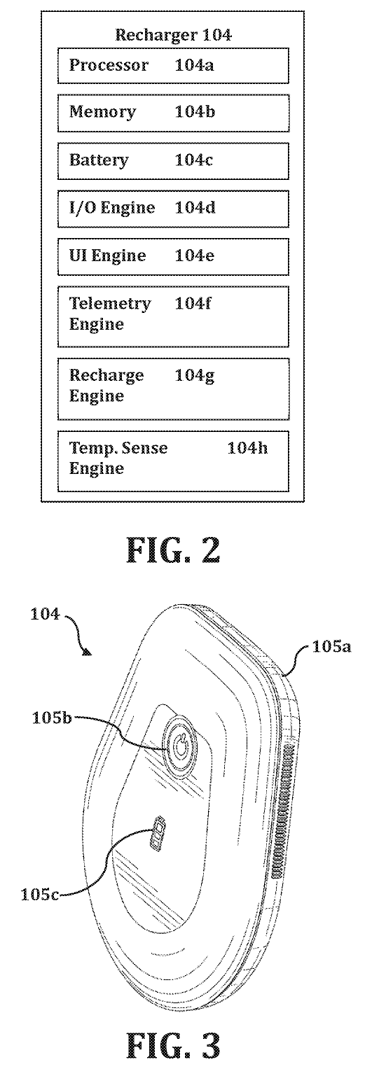

In some examples, IMD 102 can comprise a processor, a memory, a battery, a stimulation engine, a telemetry engine, a recharge engine, a temperature sensing engine, a charge sensor engine (for current, voltage), etc. for providing therapy to a patient. Such components are not depicted in FIG. 2 for ease of explanation.

Some of the subsystems of system 100 include various engines or tools, each of which is constructed, programmed, configured, or otherwise adapted, to autonomously conduct a function or set of functions. The term engine as used herein is defined as a real-world device, component, or arrangement of components implemented using hardware, such as by an application specific integrated circuit (ASIC) or field-programmable gate array (FPGA), for example, or as a combination of hardware and software, such as by a microprocessor system and a set of program instructions that adapt the engine to implement the particular functionality, which (while being executed) transform the microprocessor system into a special-purpose device. An engine can also be implemented as a combination of the two, with certain functions facilitated by hardware alone, and other functions facilitated by a combination of hardware and software.

In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations, and should generally not be limited to any particular implementation exemplified herein, unless such limitations are expressly called out. In addition, an engine can itself be composed of more than one sub-engines, each of which can be regarded as an engine in its own right. Moreover, in the examples described herein, each of the various engines corresponds to a defined autonomous functionality; however, it should be understood that in other contemplated examples, each functionality can be distributed to more than one engine. Likewise, in other contemplated examples, multiple defined functionalities may be implemented by a single engine that performs those multiple functions, possibly alongside other functions, or distributed differently among a set of engines than specifically illustrated in the examples herein.

Recharger 104 can be configured to control all aspects of a recharge session. Referring now to FIG. 2, in some examples, recharger 104 can include a processor 104a, a memory 104b, a battery 104c, an input/output (I/O) engine 104d, a user interface (UI) engine 104e, a telemetry engine 104f, a recharge engine 104g, and a temperature sense engine 104h. Recharger 104 can be configured to implement or command ETO functionality to IMD 102 using these components. Accordingly, a separate user device 106 is not required for recharging functionality or to disable IMD therapy during emergency events.

Processor 104a can be any programmable device that accepts digital data as input, is configured to process the input according to instructions or algorithms, and provides results as outputs. In some examples, processor 104acan be a central processing unit (CPU) configured to conduct the instructions of a computer program. Processor 104a is therefore configured to perform at least basic arithmetical, logical, and input and output operations. As depicted, processor 104a can be specialized for recharger 104 operation, including for implementing the ETO system. Processor 104a can also be specialized for communicator or other external instrument operation, in addition to or in place of recharger 104 operation (e.g., recharger 104 and communicator separate or together).

In some examples, processor 104a can be positioned within a housing defined by the recharger 104. In some examples, processor 104a can include instructions that, when executed, cause the processor 104a to, among other things, detect a unique input or input sequence applied to recharger, with the unique input being associated with activating ETO functionality to disable IMD therapy for a patient. The instructions can also cause processor 104a to verify that the recharger 104 is in at least one predefined operational state before activating the ETO system, and to instruct the IMD to disable therapy in response to verifying that the external instrument is in the at least one predefined operational state. In some examples, processor 104a can also include instructions that, when executed, cause the processor 104a to prevent activation of the ETO system if the unique input is not detected within a predetermined time period, and to transition the recharger 104 to an IMD reset state after therapy is disabled with the ETO system.

Memory 104b can include volatile or non-volatile memory as required by the coupled processor 104a to not only provide space to execute the instructions or algorithms, but to provide the space to store the instructions themselves. In some examples, volatile memory can include random access memory (RAM), dynamic random-access memory (DRAM), or static random-access memory (SRAM), for example. In some examples, non-volatile memory can include read-only memory, flash memory, ferroelectric RAM, hard disk, floppy disk, magnetic tape, or optical disc storage, for example. The foregoing lists in no way limit the type of memory that can be used, as these examples are not intended to limit the scope of the present disclosure.

Battery 104c can include one or more electrochemical cells to power recharger 104. Further, battery 104c can be configured to transfer or otherwise replenish power to the battery of IMD 102 when recharger 104 is communicatively or operably coupled to implantable device 102.

I/O engine 104d can be configured to communicate with the optional user device 106. In some examples, I/O engine 104d can be configured for radio, free-space optical, sonic, or electromagnetic induction communication, such as Bluetooth®, Wi-Fi, or RFID communication. In some examples, recharger 104 can include networking hardware. I/O engine 104d can be configured to operate the networking hardware to transmit and receive network communications.

UI engine 104e can be configured to present indications related to recharger 104 to a user. In some examples, the user can be a patient of IMD 102, or a clinician administering IMD 102. For example, UI engine 104e can receive commands from a firmware state machine (such as that in recharge engine 104g) to change indications on the UI. Accordingly, user interface states corresponding to the firmware state are changed. In some examples, the UI engine 104e can receive as input the command to start or stop the UI indication when the recharger 104 changes states. Accordingly, firmware states are aggregated or grouped into user interface states. Grouping of complex firmware states simplifies the user experience for the user.

In some examples, recharger 104 can include one or more visual or audible indicators (also referred to as “indicator elements”). Accordingly, UI engine 104e can include instructions to operate the indicators. For example, recharger 104 can include a single “button” or interface element surrounded by multiple LEDs that illuminate the button, and three indicators configured with a plurality of LEDs depicting a “battery,” as illustrated in FIG. 3. For example, the single button can start or stop a recharging session and change states of recharger 104. The single button can also implement the ETO system if a unique button press or sequence of button presses is provided by the user. In some examples, the unique button press or sequence may activate the ETO system only if the recharger 104 is operating in specific states. Accordingly, the ETO system can be implemented with low-UI external instruments such as recharger 104, communicators, patient remotes, and other instruments commonly used with IMD 102. In some examples, the single button has a depressed mechanical design to prevent unintended button presses.

In other examples, the recharger 104 can include a different control or interface element in addition to or in place of the button (e.g., switches, levers, sliders, toggles, dials, knobs, touch or motion sense, pressure-sensitive pads, capacitive touch surfaces, gesture controls, visual or voice sense, proximity sensors, rotary encoders, trackpads, joysticks, touchscreens, haptic feedback interfaces, biometric sensors such as fingerprint or palm readers, magnetic or RFID-based controls, smartphone or app-based controls, mechanical push-pull mechanisms, scroll wheels, soft keys, resistive touch panels, infrared sensors, piezoelectric sensors, vibration-based feedback mechanisms, or other mechanisms that a user can interact with).

In some examples, UI engine 104e can use different colored LEDs, such as green and orange. In some examples, recharger 104 can further include a speaker. Accordingly, UI engine 104e can include instructions to operate the speaker, such as with beeps or tones. Because the visual interface of recharger 104 can be hidden or difficult to see by a user operating the recharger when coupled to the patient, supplemental audio indication can be desirable.

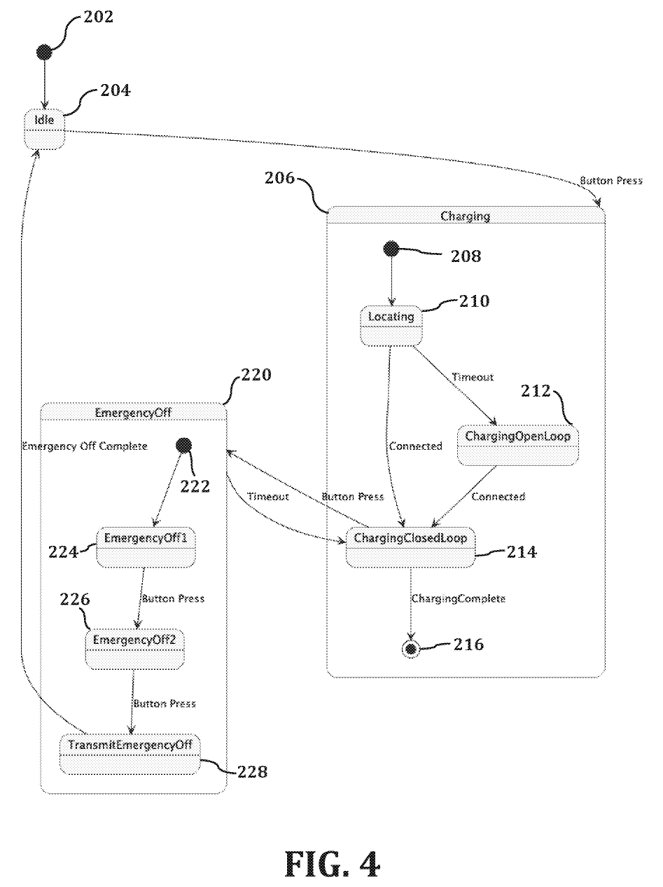

During an IMD recharge session, visual feedback can include an indication of searching for the IMD 102, IMD 102 and recharger 104 coupling interrupted, IMD 102 charge complete, alerts, and errors. These indications are described in further detail with respect to the state diagrams of FIGS. 4 through 14.

UI engine 104e can further accept input from the user to start a recharge session, change state (e.g., going to off or idle), arm the recharger for physician mode recharge, power reset the recharger (e.g., such as holding for at least 20 to 30 seconds), or to implement the ETO system to disable IMD therapy in response to an emergency event.

For patients with components or devices that have a maximum service life, UI engine 104e can be further configured to detect maximum service life and output an additional tone to indicate that a component or device has reached elective replacement. Many patients only use a charger, such as recharger 104, and not the programmer (e.g., particular software on optional user device 106), because the patient’s therapy settings never need to be changed. Additionally, some patients may carry only the recharger 104 when they leave their homes. Thus, having a maximum service life indication provides a higher probability that the user will hear the indication. In some examples, the additional tone can be placed at the beginning of recharge so as to not annoy the user. The maximum service life indication can also ensure that the recharger 104 has the capacity to implement the ETO system if an emergency arises.

Telemetry engine 104f can be configured to communicate with IMD 102. For example, data from IMD 102 can be acquired via telemetry engine 104f, which facilitates communication between the recharger 104 and the IMD 102. This communication is discussed in further detail for the first and second telemetry states 180, 190 shown in FIGS. 4 and, respectively, 12 and 13.

In some examples, telemetry engine 104f can include an antenna configured to emit the recharging field, or in other examples, two separate antennae can be used, one for communication and the other for the recharging field. In some examples, telemetry engine 104f can be configured to communicate with IMD 102 on regular intervals to obtain sensory information from the implant, such as temperature, charge current, charge voltage, etc. In some examples, the telemetry engine 104f can be configured to change durations between telemetry events with recharger 104 and IMD 102. Doing so enables recharger 104 to provide more timely feedback to users in situations where the user is actively searching for the IMD 102 (either initially or when coupling has been lost).

Recharge engine 104g can include a primary coil and drive circuitry configured to supply recharging power to IMD 102. In some examples, recharge engine 104g can further include one or more sensors to detect the relative presence of IMD 102. In some examples, one or more sensors can be configured to detect an implantable neurostimulator (“INS”) via metal detection as part of a location function. For example, one or more sensors can detect a relative degree of metal loading (high loading meaning the device is proximal the sensor; low loading meaning the device is distal the sensor). In some examples, one or more sensors can detect a reflected impedance of the INS or another IMD within the patient.

In some examples, telemetry engine 104f and recharge engine 104g can both be controlled by separate microprocessors and activate separate coils for inductive telemetry (shown in FIG. 2 as a single processor for ease of explanation). In other examples, telemetry engine 104f can interface with an RF antenna and communicate at higher frequencies (MICS band or Bluetooth® low energy (“BLE”)).

Temperature sense engine 104h can be configured with one or more temperature sensors and configured to determine a temperature of recharger 104. Recharger 104 can include other engines to determine other measurable parameters (e.g., signal strength, pressure, etc.).

FIG. 3 is a perspective view of the recharger 104. In addition to the internal components mentioned previously, recharger 104 can further include a housing 105a, a single button or interface element 105b, and a battery indicator 105c. In some examples, a ring of LEDs can be disposed around or under button 105b such that rotational displays can be created by illuminating subsequent LEDs. Battery indicator 105c can indicate the battery level of recharger 104 by, for example, illuminating with different colors to indicate the level (e.g., green for about 75% or greater charge, orange for about 25 to 75% charge, red for less than about 25% charge).

As described herein, recharger 104 can be configured to operate independently to control all aspects of a recharge session. Recharger 104 can be configured to implement the ETO system if a unique button press or sequence of button presses is provided by the patient when the recharger 104 is in certain operational states. Activating the ETO system can disable IMD therapy immediately or in a matter of seconds to resolve an emergency event. Using unique button presses or button press sequences (e.g., a given number of short button presses, or different types of button presses such as a mix of short and long presses), specifically during predefined recharger 104 or IMD 102 operational states (e.g., during closed loop recharging or when the recharger 104 is searching for the IMD 102), can eliminate or significantly reduce accidental activation. Accordingly, the ETO system can be implemented with external instruments such as recharger 104, communicators, patient remotes, and other external instruments having low-UI (e.g., only a single button or interface element).

Rechargers of the present disclosure generally have at least four distinct states during normal use: completely off, powered on and idling, locating the IMD, and charging the IMD. In rechargers with a single button or interface element, the patient is likely to press the button when the recharger is completely off to turn the recharger on, as well as when the recharger is in the idle state to start the recharging process. Pressing the button during IMD locating or charging is less likely because the patient is generally trained to start recharging and then allow the recharger to finish automatically, followed by the recharger shutting itself off when charging completes. For these reasons, the present disclosure contemplates storing the ETO system behind specific instrument operational states such as those described herein. In examples where communicators or patient remotes are used, the ETO system can be activatable when the communicator or patient remote is idling, or when they are already connected to the IMD.

The present disclosure contemplates making entry to the ETO system indistinguishable from normal instrument use until a certain number of button presses are inputted (e.g., two or more short or long presses). For example, if the recharger were in its charging state and the button was pressed, it would return to its idle state. If the ETO system were added in this path, the recharger could sit ready for the second input to activate the ETO system, but outwardly appear to be idling. If the patient did not desire to activate the ETO system, they would not input any more button presses and the recharger would simply idle after a timeout. If the patient desired to use the ETO system, they could input the next few inputs to activate the ETO system, and the recharger could then present UI showing that the ETO system is being activated.

ETO functionality can be implemented using an ETO system 220 included with the external instruments such as recharger 104, communicators, and patient handheld and therapy remotes, among others. Description of how the ETO system 220 operates to activate the ETO functionality, in conjunction with normal operating procedures of the external instruments, is described in the following sections. This description builds upon what was described previously for the arrangement between recharger 104 and IMD 102. It should be understood that ETO system 220 can be included with recharger 104, communicators, remotes, or other external instruments in various examples. FIGS. 4 through 6, which illustrate state diagrams implementing ETO system 220, will now be discussed.

FIG. 4 illustrates a first state diagram for a recharger used with various IMDs, the recharger having an ETO system 220 configured to initiate an ETO feature, according to examples of the present disclosure. The first state diagram depicts initiating the ETO feature when the recharger is operating in a closed loop charging state.

Starting from an initial operating state 202, the recharger can transition to a docked or undocked idle state 204 where the recharger awaits further instruction before transitioning to a different state. A button press or sequence of button presses, or another action against an interface element of the recharger, can cause the recharger to transition to a charging state 206. In the charging state 206, starting from an initial charging point 208, the recharger can transition to a locating state 210 to locate the IMD for charging. If a timeout is detected, such as when no telemetry feedback is received from the IMD, the recharger can transition from the locating state 210 to an open loop charging state 212. However, if the recharger locates and connects to the IMD, such as when telemetry feedback is received from the IMD, the recharger can transition to a closed loop charging state 214 to initiate closed loop charging of the IMD. The recharger can also transition from the open loop charging state 212 to the closed loop charging state 214 if the recharger subsequently locates and connects to the IMD, such as when a telemetry feedback connection is established. When closed loop charging is complete, the recharger can transition from the closed loop charging state 214 to a final charging point 216, prior to transitioning to a different operating state.

During charging at the closed loop charging state 214, the recharger can initiate the ETO system 220 using a button press or series of button presses, or another action against the interface element of the recharger. If the recharger transitions to the ETO system 220, but then subsequently experiences a timeout because of nonuse, the recharger can transition back to the closed loop charging state 214 where IMD recharging can be completed. If this happens, the recharger can transition back to the ETO system 220 if a button press or series of button presses is again detected. The process of transitioning between the closed loop charging state 214 and the ETO system 220 can be repeated until IMD charging is complete, or until no additional recharger button presses or interface actions are detected. Alternatively, the recharger could transition back to the idle state 204 if a button press or series of button presses occurs during the closed loop charging state 214, but then a subsequent button press or series of button presses are not observed after the recharger transitions to the ETO system 220.

The ETO system 220 can be initiated starting at an initial ETO state 222. The button press or series of button presses, or other recharger interface element activation during the closed loop charging state 214, can cause the recharger to transition from the initial ETO state 222 to the first ETO initiation state 224. A subsequent button press or series of button presses, or interface element activation, can cause the recharger to transition from the first ETO initiation state 224 to a second ETO initiation state 226. Similarly, a further subsequent button press or series of button presses, or interface element activation, can cause the recharger to transition from the second ETO initiation state 226 to a transmit ETO state 228. At state 228, the ETO feature can be activated to immediately or quickly turn off medical therapy from the IMD. As such, medical therapy can be conveniently disabled during emergency events or other situations that require IMD therapy to be stopped.

Complete activation of the ETO feature is concealed behind several sets of button presses or interface element activations to prevent accidental use during normal recharger operations. Additionally, according to the example illustrated in FIG. 4, the ETO system 220 can only be accessed starting at the closed loop charging state 214. This also prevents accidental use by ensuring that the ETO feature is activated only during actual emergency events or other situations where the patient intentionally desires medical therapy to stop. The ETO system 220 can be accessed from other recharger operational states in different examples, and that the ETO feature can be activated using a sequence of button presses or interface element activations as described and illustrated for closed loop charging.

After the ETO feature is activated, the recharger can transition back to the idle state 204 where the recharger awaits further instruction before transitioning to a different state. In some examples, the recharger can transmit a reset instruction to the IMD after therapy is disabled, to cause the IMD to reset prior to continued use. Accordingly, IMD operation can be temporarily disabled during emergency events before being reactivated by the patient or a clinician to resume medical therapy or recharging using the recharger.

In some examples, the button presses may be a series of button presses or interface element activations for known times, or holding a single button or interface element press for a short period of time. In some examples, the button presses or interface element activations can be characterized as short or long. In some examples, a short button press or interface element activation can last about half a second to three seconds. In some examples, a long button press or interface element activation can last greater than three seconds. A short button press or interface element activation may be longer than three seconds in other examples (i.e., a long button press or interface element activation could start at greater than about five seconds).

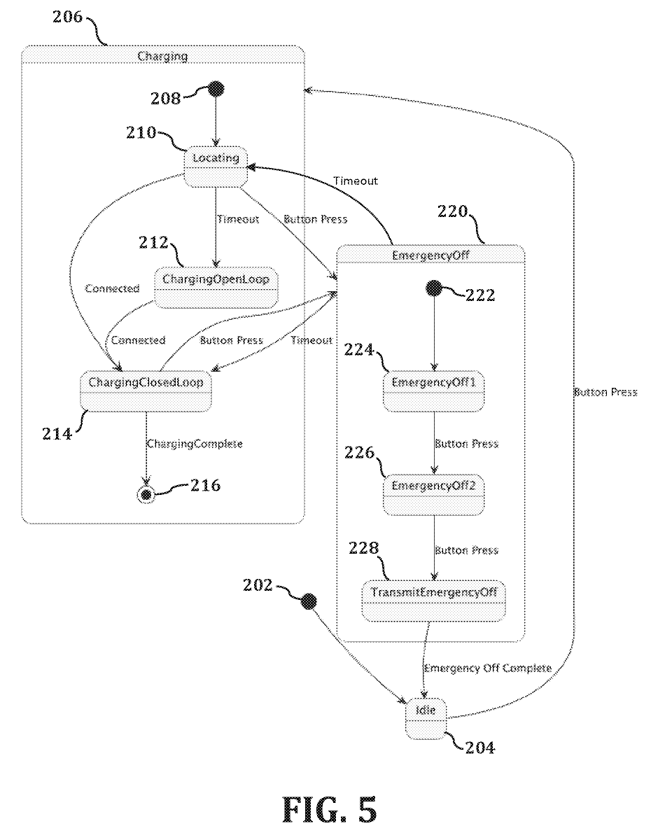

FIG. 5 illustrates a second state diagram for a recharger used with various IMDs, the recharger having an ETO system 220 configured to initiate an ETO feature, according to examples of the present disclosure. The second state diagram depicts initiating the ETO feature when the recharger is operating in a charging locating state. This represents one alternative example of the recharger accessing and initiating the ETO system 220 to disable medical therapy during emergency events.

Like the first state diagram of FIG. 4, starting from the initial charging point 208 in the charging state 206, the recharger can transition to the locating state 210 to locate the IMD for charging. If a timeout is detected, the recharger can transition from the locating state 210 to the open loop charging state 212. However, if the recharger locates and connects to the IMD, the recharger can transition to the closed loop charging state 214 to initiate closed loop charging. The recharger can also transition from the open loop charging state 212 to the closed loop charging state 214 if the recharger subsequently locates and connects to the IMD. When closed loop charging is complete, the recharger can transition from the closed loop charging state 214 to the final charging point 216, prior to transitioning to a different operating state.

Also like the first state diagram of FIG. 4, during closed loop charging at the closed loop state 214, the recharger can initiate the ETO system 220 using a button press or series of button presses, or another action against an interface element of the recharger. If the recharger transitions to the ETO system 220, but then subsequently experiences a timeout because of nonuse, the recharger can transition back to the closed loop state 214 where IMD recharging can be completed. If this happens, the recharger can transition back to the ETO system 220 if a button press or series of button presses, or other activation of a recharger interface element, is again detected. The process of transitioning to and from the closed loop state 214 and the ETO system 220 can be repeated until IMD charging is complete or no additional recharger button presses or interface element activations are detected. Alternatively, if the recharger transitions to the ETO system 220 from the closed loop state 214, but then subsequently experiences a timeout, the recharger can transition back to the locating state 210 to resume locating the IMD for charging. The recharger can then proceed through the second state diagram as described previously.

The ETO system 220 according to the second state diagram can be initiated using a similar process as what is described with respect to the first state diagram. Specifically, the ETO system 220 can be initiated starting at an initial ETO state 222, followed by transition to the first ETO initiation state 224. The recharger can transition to the second ETO initiation state 226 and the transmit ETO state 228 each in response to a button press or a series of button presses, or other interface element activations. At the transmit ETO state 228, the ETO feature can be activated which immediately or quickly turns off medical therapy from the IMD. After the ETO feature is activated, the recharger can transition back to the idle state 204 where the recharger awaits further instruction before transitioning to a different state. Additionally, the recharger according to the second state diagram can transition directly from the initial operating state 202 to the idle state 204 if the recharger is not acted upon by the patient or clinician, or is not otherwise activated.

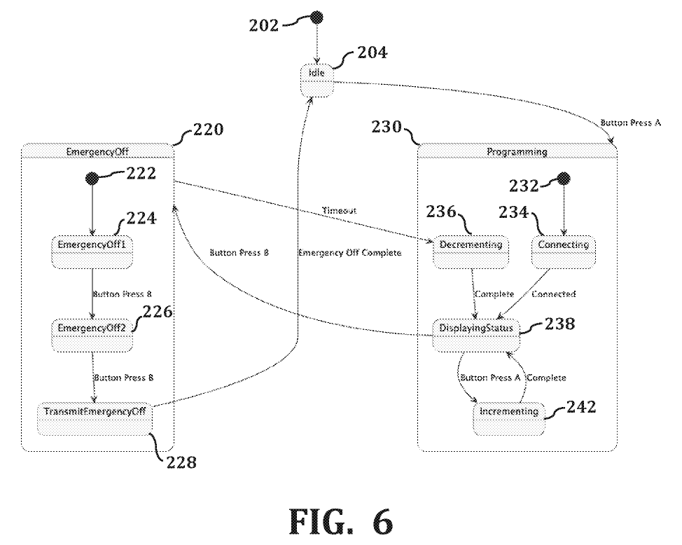

FIG. 6 illustrates a third state diagram for a patient handheld or therapy remote used with various IMDs, the remote having an ETO system 220 configured to initiate an ETO feature, according to examples of the present disclosure. The patient handheld or therapy remote can be configured to control operation of the IMD, including adjusting the therapy provided by the IMD to the patient. The third state diagram depicts initiating the ETO feature from a programming state of the patient remote. This represents a different external instrument that the ETO system 220 is compatible with to disable medical therapy during emergency events (including situations where a recharger also including the ETO system 220 is not available).

Starting from the initial operating state 202, the recharger can transition to an idle state 204 to await further instruction before transitioning to a different state. A first button press or sequence of button presses, or another first action against an interface element of the recharger, can cause the recharger to transition to a programming state 230. In the programming state 230, starting from an initial programming point 232, the recharger can transition to a connecting state 234 where it attempts to connect with the IMD, for example establishing a telemetry connection with the IMD. When a connection is established, the recharger can transition to a displaying status state 238 where the recharger displays its current operational status. A subsequent first button press or sequence of button presses, or another first action against a recharger interface element, can cause the recharger to transition to an incrementing state 242. When recharger incrementing is complete at state 242, the recharger can transition back to the displaying status state 242.

Alternatively, a second button press or sequence of button presses, or another second action against the interface element, can cause the recharger to initiate the ETO system 220. The ETO system 220 according to the third state diagram can be initiated using a similar process as described with respect to the first and second state diagrams. Specifically, the ETO system 220 can be initiated starting at an initial ETO state 222, followed by transition to the first ETO initiation state 224. The recharger can transition to the second ETO initiation state 226 and the transmit ETO state 228 each in response to a second button press or a series of second button presses, or a second interface element activation. At the transmit ETO state 228, the ETO feature can be activated which immediately or quickly turns off medical therapy from the IMD. After therapy is turned off or otherwise disabled, the recharger can transition from the transmit ETO state 228 back to the idle state 204 to await further instructions for operation.

Similar state diagrams as those described previously can be used to illustrate an ETO system 220 for a communicator or another external low-UI instrument commonly used with IMDs. All or most of the previous description can be applied to communicators and other external instruments, in addition to the wireless rechargers and patient remotes that provide the focus for the illustrative figures and accompanying description.

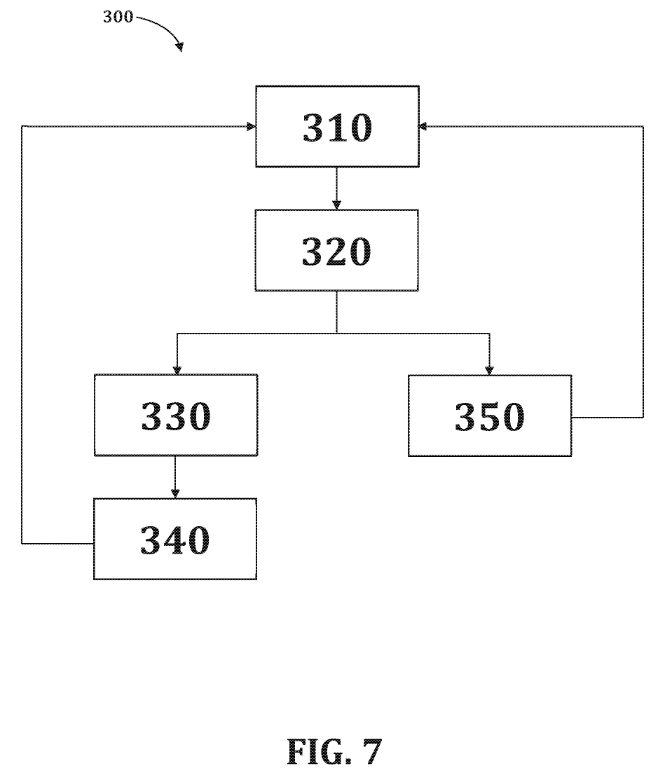

FIG. 7 is a method 300 of operating an emergency therapy off system for use with medical devices, according to examples of the present disclosure. Method 300 may be used by emergency therapy off system 220 included with recharger 104, communicators, remotes, or other external instruments operably or communicatively couplable to an IMD such as IMD 102 (e.g., neurostimulators, pacemakers, drug delivery devices, other IMDs that use external low-UI instruments). In some examples, method 300 can be implemented on a non-transitory computer-readable medium encoding instructions operable to cause at least one processor of an external instrument for use with an IMD to perform the operations described below.

At 310, method 300 can include detecting a unique input or input sequence applied to the interface element, wherein the unique input or input sequence is associated with activating an emergency therapy off (“ETO”) feature to disable IMD therapy for a patient. The unique input or input sequence may involve a combination of short or long presses to a button, or activations of an interface element, on an external instrument such as a recharger or a communicator.

At 320, method 300 can include verifying that the external instrument is in at least one predefined operational state before activating the ETO feature. As discussed previously, the at least one predefined operational state can include a closed loop state and an IMD locating state for rechargers, and an idling state or an already connected to IMD state for communicators and patient remotes, or vice versa.

At 330, method 300 can include instructing the IMD to disable therapy in response to verifying that the external instrument is in the at least one predefined operational state. This can be achieved using the ETO feature that is generally activatable only when one or more unique button presses or sequences of button presses is provided to the external instrument. Additionally, this operation will generally only occur if the external instrument is in the at least one predefined operational state as verified at 320.

At 340, method 300 can include transitioning the external instrument to an IMD reset state after therapy is disabled at 330. The external instrument generally will not operate until after the IMD is reset or turned back on. In some examples, the transition may occur after the IMD receives a command from the external instrument. The command may be at least one of an emergency off command, a battery switch open command, a therapy off command, an amplitude-to-zero command, or similar commands that disable IMD operation. The command may disable the IMD for a period of time, such as one hour, 8 hours, 12 hours, 24 hours, one day, one week, or other periods of time less than or greater than these values.

If verification at 320 fails, then at 350 method 300 can include preventing activation of the ETO feature if the unique input or input sequence is not detected within a predetermined time period. The predetermined time period may be a few seconds, a minute, or a time period in between in some examples.

Accordingly, method 300 can be used to activate the ETO feature with an IMD to disable therapy if an emergency event is present. The emergency event can be the patient experiencing pain during therapy in some examples. The ETO feature can only be activated if a unique combination or instance of button presses is provided, and the external instrument is in a specific operational state. Method 300 can be used with external instruments such as rechargers and communicators having minimal UI (e.g., a single button or interface element). Patients are provided with ease of mind knowing that they can activate the ETO feature using just the external instrument rather than other devices, such as a patient handset or a magnet, normally required to deactivate therapy in emergencies.

According to a first example of the present disclosure, an external instrument for use with an IMD is provided. The external instrument can include a housing with an interface element, at least one processor inside the housing, and instructions that, when executed on the at least one processor, can cause the at least one processor to: detect a unique input applied to the interface element, wherein the unique input is associated with activating an ETO feature to disable IMD therapy for a patient; verify that the external instrument is in at least one predefined operational state before activating the ETO feature; and instruct the IMD to disable therapy in response to verifying that the external instrument is in the at least one predefined operational state.

In a second example of the first example or any other example, the at least one processor can be configured to enable recharger operation to recharge the IMD.

In a third example of the first example or any other example, the at least one processor can be configured to enable communicator operation to communicate with the IMD.

In a fourth example of the first example or any other example, the at least one predefined operational state can include a closed loop state, an IMD locating state, an idling state, and an already connected to IMD state.

In a fifth example of the first example or any other example, the unique input can include a predetermined number of interactions with the interface element.

In a sixth example of the first example or any other example, the unique input can include a combination of short interactions and long interactions with the interface element, with a short interaction lasting approximately 0.5 to 5 seconds, and a long interaction exceeding approximately 5 seconds.

In a seventh example of the first example or any other example, the external instrument can further include an indicator element configured to confirm activation of the ETO feature.

In an eighth example of the first example or any other example, the external instrument can further include an instruction that, when executed on the at least one processor, can cause the at least one processor to: prevent activation of the ETO feature if the unique input is not detected within a predetermined time period.

In a ninth example of the first example or any other example, the external instrument can further include an instruction that, when executed on the at least one processor, can cause the at least one processor to: transition to an IMD reset state after therapy is disabled with the ETO feature.

According to a tenth example of the present disclosure, a medical treatment system is provided. The system can include an IMD configured to provide a medical therapy for a patient, and an external instrument coupled to the IMD. The external instrument can include a housing with an interface element, at least one processor, and instructions that, when executed on the at least one processor, can cause the at least one processor to: detect a unique input applied to the interface element, wherein the unique input is associated with activating an ETO feature to disable IMD therapy for a patient; verify that the external instrument is in at least one predefined operational state before activating the ETO feature; and instruct the IMD to disable therapy in response to verifying that the external instrument is in the at least one predefined operational state.

In an eleventh example of the tenth example or any other example, the external instrument can be a recharger configured to charge the IMD.

In a twelfth example of the tenth example or any other example, the external instrument can be a communicator configured to communicate with the IMD.

In a thirteenth example of the tenth example or any other example, the IMD can be an implantable neurostimulator.

In a fourteenth example of the tenth example or any other example, the interface element can be a button provided on the housing.

In a fifteenth example of the tenth example or any other example, the at least one predefined operational state can include one or more of a closed loop state, an IMD locating state, an idling state, and an already connected to IMD state.

In a sixteenth example of the tenth example or any other example, the unique input can include at least one of a predetermined number of interactions with the interface element; and a combination of short interactions and long interactions with the interface element, wherein a short interaction can last approximately 0.5 to 5 seconds, and a long interaction can exceed approximately 5 seconds.

In a seventeenth example of the tenth example or any other example, the system can further include an indicator element configured to confirm activation of the ETO feature.

In an eighteenth example of the tenth example or any other example, the system can further include an instruction that, when executed on the at least one processor, can cause the at least one processor to: prevent activation of the ETO feature if the unique input is not detected within a predetermined time period.

In a nineteenth example of the tenth example or any other example, the system can further include an instruction that, when executed on the at least one processor, can cause the at least one processor to: transition to an IMD reset state after therapy is disabled with the ETO feature.

In a twentieth example of the tenth example or any other example, the external instrument can be a patient handheld remote configured to control the IMD.

In a twenty-first example of the tenth example or any other example, the external instrument can be an electronic device configured to control the IMD via a patient application.

According to a twenty-second example of the present disclosure, a method or a non-transitory computer-readable medium encoding instructions operable to cause at least one processor of an external instrument for use with an IMD can be provided. The method or the instructions can include detecting a unique input applied to the interface element, with the unique input generally being associated with activating an ETO feature to disable IMD therapy for a patient; verifying that the external instrument is in at least one predefined operational state before activating the ETO feature; and instructing the IMD to: disable therapy in response to verifying that the external instrument is in the at least one predefined operational state, and transition to an IMD reset state after therapy is disabled with the ETO feature; or prevent activation of the ETO feature if the unique input is not detected within a predetermined time period.

Rechargers compatible with the present disclosure include those provided in commonly assigned U.S. Patent No. 11,944,833 to Fried et al., the disclosure of which is incorporated by reference herein in its entirety.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structures suitable for implementation of the described techniques. Also, the techniques could be fully implemented in one or more circuits or logic elements.

The techniques of this disclosure may also be described in the following examples.

Example 1: An external instrument for use with an implantable medical device (IMD), comprising: a housing with an interface element; at least one processor inside the housing; and instructions that, when executed on the at least one processor, cause the at least one processor to:detect a unique input applied to the interface element, wherein the unique input is associated with activating an emergency therapy off (ETO) feature to disable IMD therapy for a patient; verify that the external instrument is in at least one predefined operational state before activating the ETO feature; and instruct the IMD to disable therapy in response to verifying that the external instrument is in the at least one predefined operational state.

Example 2: The external instrument of Example 1, wherein the at least one processor is configured to enable recharger operation to recharge the IMD.

Example 3: The external instrument of any one of Examples 1-2, wherein the at least one processor is configured to enable communicator operation to communicate with the IMD.

Example 4: The external instrument of any one of Examples 1-3, wherein the at least one predefined operational state comprises a closed loop state, an IMD locating state, an idling state, and an already connected to IMD state.

Example 5: The external instrument of any one of Examples 1-4, wherein the unique input comprises a predetermined number of interactions with the interface element.

Example 6: The external instrument of any one of Examples 1-5, wherein the unique input comprises a combination of short interactions and long interactions with the interface element, wherein a short interaction lasts approximately 0.5 to 5 seconds, and a long interaction exceeds approximately 5 seconds.

Example 7: The external instrument of any one of Examples 1-6, further comprising an indicator element configured to confirm activation of the ETO feature.

Example 8: The external instrument of any one of Examples 1-7, further comprising an instruction that, when executed on the at least one processor, causes the at least one processor to: prevent activation of the ETO feature if the unique input is not detected within a predetermined time period.

Example 9: The external instrument of any one of Examples 1-8, further comprising an instruction that, when executed on the at least one processor, causes the at least one processor to: transition to an IMD reset state after therapy is disabled with the ETO feature.

Example 10: A medical treatment system, comprising: an implantable medical device (IMD) configured to provide a medical therapy for a patient; an external instrument coupled to the IMD and comprising: a housing with an interface element; at least one processor; and instructions that, when executed on the at least one processor, cause the at least one processor to: detect a unique input applied to the interface element, wherein the unique input is associated with activating an emergency therapy off (ETO) feature to disable IMD therapy for a patient; verify that the external instrument is in at least one predefined operational state before activating the ETO feature; and instruct the IMD to disable therapy in response to verifying that the external instrument is in the at least one predefined operational state.

Example 11: The system of Example 10, wherein the external instrument is a recharger configured to charge the IMD.

Example 12: The system of Example 10, wherein the external instrument is a communicator configured to communicate with the IMD.

Example 13: The system of Example 10, wherein the external instrument is a patient handheld remote configured to control the IMD.

Example 14: The system of Example 10, wherein the external instrument is an electronic device configured to control the IMD via a patient application.

Example 15: The system of any one of Examples 10-14, wherein the IMD is an implantable neurostimulator.

Example 16: The system of any one of Examples 10-15, wherein the interface element is a button provided on the housing.

Example 17: The system of any one of Examples 10-16, wherein the at least one predefined operational state comprises a closed loop state, an IMD locating state, an idling state, and an already connected to IMD state.

Example 18: The system of any one of Examples 10-17, wherein the unique input comprises at least one of: a predetermined number of interactions with the interface element; and a combination of short interactions and long interactions with the interface element, wherein a short interaction lasts approximately 0.5 to 5 seconds, and a long interaction exceeds approximately 5 seconds.

Example 19: The system of any one of Examples 10-18, further comprising an indicator element configured to confirm activation of the ETO feature.

Example 20: The system of any one of Examples 10-19, further comprising an instruction that, when executed on the at least one processor, causes the at least one processor to: prevent activation of the ETO feature if the unique input is not detected within a predetermined time period.

Example 21: The system of any one of Examples 10-20, further comprising an instruction that, when executed on the at least one processor, causes the at least one processor to: transition to an IMD reset state after therapy is disabled with the ETO feature.

Example 22: A non-transitory computer-readable medium encoding instructions operable to cause at least one processor of an external instrument for use with an implantable medical device (IMD) to: detect a unique input applied to an interface element, wherein the unique input is associated with activating an emergency therapy off (ETO) feature to disable IMD therapy for a patient; verify that the external instrument is in at least one predefined operational state before activating the ETO feature; and instruct the IMD to: disable therapy in response to verifying that the external instrument is in the at least one predefined operational state, and transition to an IMD reset state after therapy is disabled with the ETO feature; or prevent activation of the ETO feature if the unique input is not detected within a predetermined time period.

Various examples of the disclosure have been described. These and other examples are within the scope of the following claims.

1. An external instrument for use with an implantable medical device (IMD), comprising:
a housing with an interface element;
at least one processor inside the housing; and
instructions that, when executed on at least one processor, cause at least one processor to:
detect a unique input applied to the interface element, wherein the unique input is associated with activating an emergency therapy off (ETO) feature to disable IMD therapy for a patient;
verify that the external instrument is in at least one predefined operational state before activating the ETO feature; and
instruct the IMD to disable therapy in response to verifying that the external instrument is in at least one predefined operational state.

2. The external instrument of claim 1, wherein at least one processor is configured to enable recharger operation to recharge the IMD.

3. The external instrument of claim 1, wherein at least one processor is configured to enable communicator operation to communicate with the IMD.

4. The external instrument of claim 1, wherein at least one predefined operational state comprises a closed loop state, an IMD locating state, an idling state, and an already connected to IMD state.

5. The external instrument of claim 1, wherein the unique input comprises a predetermined number of interactions with the interface element.

6. The external instrument of claim 1, wherein the unique input comprises a combination of short interactions and long interactions with the interface element, wherein a short interaction lasts approximately 0.5 to 5 seconds, and a long interaction exceeds approximately 5 seconds.

7. The external instrument of claim 1, further comprising an indicator element configured to confirm activation of the ETO feature.

8. The external instrument of claim 1, further comprising an instruction that, when executed on at least one processor, causes at least one processor to:
prevent activation of the ETO feature if the unique input is not detected within a predetermined time period.

9. The external instrument of claim 1, further comprising an instruction that, when executed on at least one processor, causes at least one processor to:
transition to an IMD reset state after therapy is disabled with the ETO feature.

10. A medical treatment system, comprising:
an implantable medical device (IMD) configured to provide a medical therapy for a patient;
an external instrument coupled to the IMD and comprising:
a housing with an interface element;
at least one processor; and
instructions that, when executed on at least one processor, cause at least one processor to:
detect a unique input applied to the interface element, wherein the unique input is associated with activating an emergency therapy off (ETO) feature to disable IMD therapy for a patient;
verify that the external instrument is in at least one predefined operational state before activating the ETO feature; and
instruct the IMD to disable therapy in response to verifying that the external instrument is in at least one predefined operational state.

11. The system of claim 10, wherein the external instrument is a recharger configured to charge the IMD.

12. The system of claim 10, wherein the external instrument is a communicator configured to communicate with the IMD.

13. The system of claim 10, wherein the external instrument is a patient handheld remote configured to control the IMD.

14. The system of claim 10, wherein the external instrument is an electronic device configured to control the IMD via a patient application.

15. The system of claim 10, wherein the interface element is a button provided on the housing.

16. The system of claim 10, wherein at least one predefined operational state comprises a closed loop state, an IMD locating state, an idling state, and an already connected to IMD state.

17. The system of claim 10, wherein the unique input comprises at least one of:
a predetermined number of interactions with the interface element; and
a combination of short interactions and long interactions with the interface element, wherein a short interaction lasts approximately 0.5 to 5 seconds, and a long interaction exceeds approximately 5 seconds.

18. The system of claim 10, further comprising an instruction that, when executed on at least one processor, causes at least one processor to:
prevent activation of the ETO feature if the unique input is not detected within a predetermined time period.

19. The system of claim 10, further comprising an instruction that, when executed on at least one processor, causes at least one processor to:
transition to an IMD reset state after therapy is disabled with the ETO feature.

20. A non-transitory computer-readable medium encoding instructions operable to cause at least one processor of an external instrument for use with an implantable medical device (IMD) to:
detect a unique input applied to the interface element, wherein the unique input is associated with activating an emergency therapy off (ETO) feature to disable IMD therapy for a patient;
verify that the external instrument is in at least one predefined operational state before activating the ETO feature; and
instruct the IMD to:
disable therapy in response to verifying that the external instrument is in the at least one predefined operational state, and transition to an IMD reset state after therapy is disabled with the ETO feature; or
prevent activation of the ETO feature if the unique input is not detected within a predetermined time period.
